(19) United States
(12) Patent Application Publication
(10) Pub. No.: US 2026/0165570 A1
(43) Pub. Date: Jun. 18, 2026

(54) ENDOSCOPE REPROCESSOR, CONTROL METHOD, AND CONTROL APPARATUS FOR ENDOSCOPE CONDUIT CLEANING

(71) Applicant: OLYMPUS MEDICAL SYSTEMS CORP., Tokyo (JP)

(72) Inventor: Shio NOGAWA, Tokyo (JP)

(73) Assignee: OLYMPUS MEDICAL SYSTEMS CORP., Tokyo (JP)

(21) Appl. No.: 19/532,181

(22) Filed: Feb. 6, 2026

Related U.S. Application Data
(63) Continuation of application No. PCT/JP2023/029256, filed on Aug. 10, 2023.

(51) Int. Cl. A61B 1/12; A61B 1/00

(52) U.S. Cl. CPC A61B 1/12; A61B 1/00045; A61B 1/00128

(57) ABSTRACT

An endoscope reprocessor includes a first tube connectable to a first ferrule of an endoscope, a second tube connectable to a second ferrule, a fluid supply assembly, a sensor, and a processor. The fluid supply assembly supplies fluid to the endoscope's internal conduit via the first and second tubes. The internal conduit includes a first conduit communicating with the first ferrule, a second conduit communicating with the second ferrule, and a third conduit, all merging at a merging point. The processor performs a first cleaning process to supply, when the first tube is disconnected from the first ferrule, fluid to the first and third conduits via the second conduit and the second tube connected to the second ferrule, determine an end timing of the first cleaning process based on a detection signal from the sensor, and perform a second cleaning process to supply fluid to the first and second tubes.

### CROSS REFERENCE TO RELATED APPLICATION

This application is a continuation of International Application No. PCT/JP2023/029256, filed on Aug. 10, 2023, the entire contents of which are incorporated herein by reference.

### FIELD

The present disclosure relates to an endoscope reprocessor used for endoscope reprocessing, a control method implementable by the endoscope reprocessor used for the endoscope reprocessing, and a control apparatus.

### BACKGROUND

For endoscopes used in the medical field, reprocessing, such as cleaning and disinfection, is essential for reuse after an insertion portion is inserted into the body to perform in-vivo observation and treatment with a treatment instrument. An endoscope reprocessor is used to perform reprocessing safely, reliably, and automatically.

An endoscope reprocessor removes contaminants such as blood clots and mucus adhering to internal conduits of the endoscope by connecting a connector of a tube to each of a suction ferrule and a forceps ferrule of the endoscope, supplying fluid to each of the tubes, and discharging the fluid from an opening at a distal end of the insertion portion.

International Publication No. WO 2015/001843 discloses an endoscope cleaning apparatus that cleans a forceps ferrule using liquid that leaks out from a space between the forceps ferrule and a connector of a tube connected to the forceps ferrule.

International Publication No. WO 2016/194456 discloses an endoscope reprocessor that performs flow control to adjust a flow rate of a first fluid supply unit that supplies liquid as a fluid and a flow rate of a second fluid supply unit that supplies gas as a fluid.

### SUMMARY

According to aspects of the present disclosure, an endoscope reprocessor is provided, which includes a first tube, a second tube, a fluid supply assembly, a sensor, and a processor. The first tube is connectable to a first ferrule of an endoscope. The second tube is connectable to a second ferrule of the endoscope. The fluid supply assembly is configured to supply a fluid to an internal conduit of the endoscope via the first and second tubes. The internal conduit includes a first conduit communicating with the first ferrule, a second conduit communicating with the second ferrule, and a third conduit. Each of the first to third conduits is open at one end and merges with the other two conduits at a merging point at another end. The sensor is configured to detect a property of the fluid. The processor is configured to perform a first cleaning process to cause the fluid supply assembly to supply, when the first tube is not connected to the first ferrule, the fluid to the first conduit and the third conduit via the second conduit and the second tube connected to the second ferrule. The processor is further configured to determine an end timing of the first cleaning process based on a detection signal from the sensor. The processor is further configured to perform a second cleaning process to cause the fluid supply assembly to supply the fluid to the first and second tubes.

According to aspects of the present disclosure, further provided is a control method implementable by an endoscope reprocessor. The reprocessor includes a first tube connectable to a first ferrule of an endoscope, and a second tube connectable to a second ferrule of the endoscope. The control method includes performing a first cleaning process to supply, when the first tube is not connected to the first ferrule, a fluid to a first conduit and a third conduit via a second conduit and the second tube connected to the second ferrule. The endoscope includes an internal conduit including the first conduit communicating with the first ferrule, and the second conduit communicating with the second ferrule. The first and second conduits converge into the third conduit having an opening at a distal end of an insertion portion of the endoscope. The control method further includes determining an end timing of the first cleaning process based on a detection signal from a sensor configured to detect a property of the fluid. The control method further includes performing a second cleaning process to supply the fluid to the first and second tubes.

According to aspects of the present disclosure, further provided is a control apparatus that includes a connection interface and a processor. The connection interface is connectable to a fluid supply assembly. The fluid supply assembly is configured to supply a fluid to an internal conduit of an endoscope via a first tube and a second tube. The endoscope includes a first ferrule connectable to the first tube, a second ferrule connectable to the second tube, and the internal conduit. The internal conduit includes a first conduit communicating with the first ferrule, and a second conduit communicating with the second ferrule. The first and second conduits converge into a third conduit having an opening at a distal end of an insertion portion of the endoscope. The processor is configured to perform a first cleaning process to cause the fluid supply assembly to supply, when the first tube is not connected to the first ferrule, a fluid to the first conduit and the third conduit via the second conduit and the second tube connected to the second ferrule. The processor is further configured to determine an end timing of the first cleaning process based on a detection signal from a sensor configured to detect a property of the fluid. The processor is further configured to perform a second cleaning process to cause the fluid supply assembly to supply the fluid to the first and second tubes.

### BRIEF DESCRIPTION OF DRAWINGS

FIG. 1 is a perspective view of an endoscope reprocessor according to an embodiment.

FIG. 2 is a configuration diagram of a main part of the endoscope reprocessor according to the embodiment.

FIG. 3 is a cross-sectional view of a joint part between a connector of a tube and a ferrule of a forceps hole in the endoscope reprocessor according to the embodiment.

FIG. 4 is a cross-sectional view of a merging section of conduits in the endoscope reprocessor according to the embodiment.

FIG. 5 is a flowchart of a method of operating the endoscope reprocessor according to the embodiment.

FIG. 6 is a cross-sectional view of the merging section of the conduits in the endoscope reprocessor according to the embodiment.

FIG. 7 is a cross-sectional view of the merging section of the conduits in the endoscope reprocessor according to the embodiment.

### DESCRIPTION OF EMBODIMENTS

An endoscope reprocessor 1 of an embodiment of the present disclosure will be described below using the drawings. Hereinafter, the endoscope reprocessor 1 may be referred to as a reprocessor 1.

It should be noted that the drawings based on the embodiment are schematic. The relationship between the thickness and width of each part and the ratio of the thickness of each part differ from the actual ones. The drawings also contain parts in which dimensional relationships and ratios are different from each other.

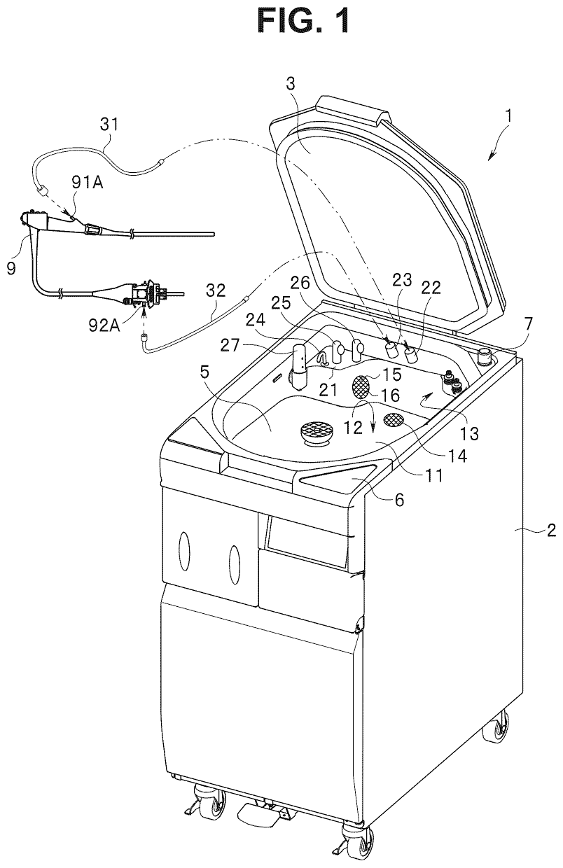

As shown in FIG. 1, the reprocessor 1 includes a main body 2 and a top cover 3 which is openable and closable. FIG. 1 shows a state where the top cover 3 of the reprocessor 1 is open.

The reprocessor 1 is an apparatus for performing reprocessing (restoration processing) on an endoscope 9 or an endoscope accessory. The reprocessing may be cleaning to clear away contaminants such as organic matter, disinfection to inactivate predetermined microorganisms, sterilization to eliminate or kill all microorganisms, or any combination of these.

The main body 2 includes, at the top thereof, a processing tank 5 for performing processing such as cleaning and disinfection of the endoscope 9, an operation panel 6, and a water-supply-hose connection port 7.

The processing tank 5 stores a liquid such as a cleaning solution, water, an alcohol disinfectant solution, or a sterilization solution. The processing tank 5 includes an endoscope placement section 11 and a tray 21.

The endoscope placement section 11 includes a bottom surface 12 and a side surface 13, is configured to receive the endoscope 9 thereon, and stores liquid. A discharge port 14 for discharging the stored liquid is provided in the bottom surface 12 of the endoscope placement section 11. A circulation port 16 having a mesh filter 15 is provided in the side surface 13 of the endoscope placement section 11. The circulation port 16 is in communication with a liquid pump 51 mentioned below. The circulation port 16 may be provided in the bottom surface 12.

The tray 21 is provided at a position adjacent to the endoscope placement section 11 and higher than the endoscope placement section 11. The tray 21 includes a water supply port 22, a gas feeding port 23, a cleaning solution nozzle 24, a disinfectant solution nozzle 25, a water supply nozzle 26, and a water level sensor 27.

The water supply port 22 is a port to which a first tube 31 is to be connected. The gas feeding port 23 is a port to which a second tube 32 is to be connected. The number of ports that the reprocessor 1 has is not limited to 2. It should be noted that the water supply port 22 may have the function of the gas feeding port 23, and the gas feeding port 23 may have the function of the water supply port 22.

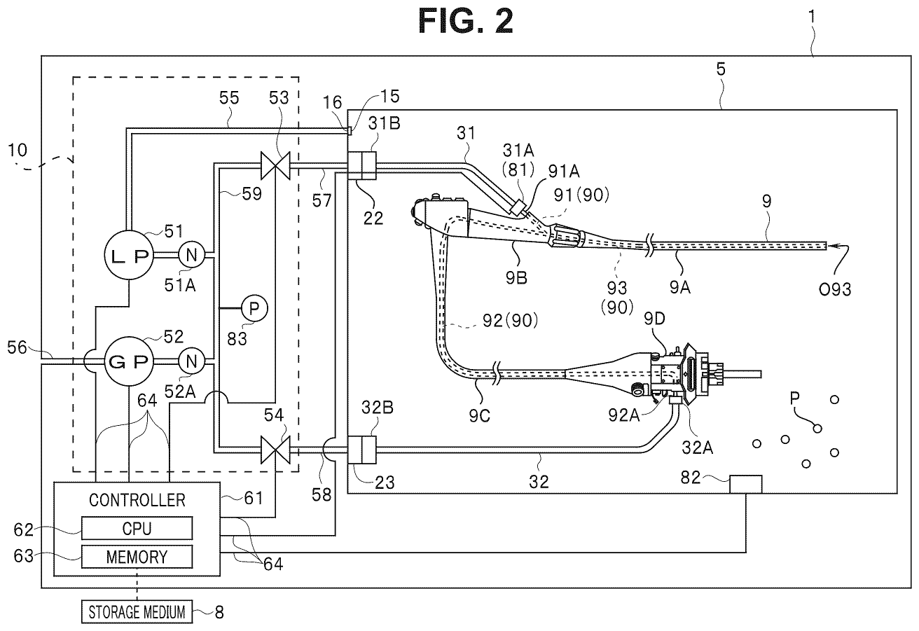

The cleaning solution nozzle 24 supplies a cleaning solution to the processing tank 5. The disinfectant solution nozzle 25 supplies a disinfectant solution to the processing tank 5. The water supply nozzle 26 supplies water taken in from the water-supply-hose connection port 7 to the processing tank 5, and also supplies the liquid in the processing tank 5 taken in from the circulation port 16 with the mesh filter 15 back to the processing tank 5, thereby circulating the liquid. The mesh filter 15 filters contaminants P from the liquid (see FIG. 2). The water level sensor 27 detects a water level of the liquid stored in the processing tank 5. The water level sensor 27 may be installed on the bottom surface 12.

The operation panel 6 is placed at an upper front section of the main body 2. The operation panel 6 includes various operation buttons and a display panel, which are not shown. A user uses the operation panel 6 to give various instructions to the reprocessor 1.

The water-supply-hose connection port 7 is provided at an upper rear section of the main body 2. A water supply hose connected to an unshown water faucet is connected to the water-supply-hose connection port 7, and water is supplied to the reprocessor 1 via the water supply nozzle 26.

The top cover 3 is provided openably and closably on the top of the main body 2. In the reprocessor 1, by bringing the top cover 3 into an opened state, the endoscope 9 can be placed on the endoscope placement section 11, and connection between the endoscope 9 and the reprocessor 1 can be established by means of the first tube 31 and the second tube 32. The reprocessor 1 is in a state where reprocessing is possible by bringing the top cover 3 into a closed state after the endoscope 9 is set.

FIG. 2 shows a state where the endoscope 9 to which the first tube 31 and the second tube 32 are connected is housed in the reprocessor 1. It should be noted that FIG. 2 shows only the main configuration of the present disclosure. The reprocessor 1 may have a configuration different from that shown in FIG. 2 as long as it has the same functions as the configuration shown in FIG. 2.

The endoscope 9 includes an insertion portion 9A to be inserted into a body, an operation portion 9B, a universal cord 9C, and an endoscope connector 9D. The endoscope 9 includes a conduit (internal conduit) 90 inside thereof. The conduit 90 includes a first conduit 91, a second conduit 92, and a third conduit 93. One end of the first conduit 91 is provided with a forceps ferrule 91A (a first ferrule), and the other end merges with the second conduit 92 at a merging section (merging point). One end of the second conduit 92 is provided with a gas feeding ferrule (a second ferrule, hereinafter may be referred to as a gas supply ferrule) 92A, and the other end merges with the first conduit 91 at the merging section (merging point). In other words, the first conduit 91 is in communication with the forceps ferrule 91A, and the second conduit 92 is in communication with the gas feeding ferrule 92A.

The third conduit 93, where the first conduit 91 and the second conduit 92 merge, passes through the insertion portion 9A and has an opening O93 at a distal end (i.e., the first conduit 91 and the second conduit 92 converge into the third conduit 93 having an opening O93 at a distal end of the insertion portion 9A). The first conduit 91 and the third conduit 93 configure a forceps channel. A treatment instrument such as a forceps inserted from the forceps ferrule 91A passes through the first conduit 91 and the third conduit 93, and a distal end of the treatment instrument protrudes from the opening O93. Fluid fed from the gas feeding ferrule 92A is emitted from the opening O93 of the insertion portion 9A via the second conduit 92 and the third conduit 93. It should be noted that, when the endoscope 9 is used, the gas feeding ferrule 92A is not used exclusively for feeding gas, but is also used for suction, feeding water, or feeding gas-liquid mixed water.

In other words, the endoscope 9 includes the internal conduit 90 including the first conduit 91, the second conduit 92, and the third conduit 93. The first conduit 91, the second conduit 92, and the third conduit 93 each open on one end side, and merge at the merging point on another end side.

The first tube 31 includes a first connector 31A provided at one end thereof and connected to the forceps ferrule 91A, which is a first ferrule of the endoscope 9, and a connector 31B provided at the other end thereof and connected to the water supply port 22 of the reprocessor 1. The water supply port 22 is in communication with the forceps ferrule 91A of the endoscope 9 via the first tube 31.

The first connector 31A is provided with a first sensor (hereinafter may be referred to as a connection sensor) 81 that detects connection to the forceps ferrule 91A.

The second tube 32 includes a second connector 32A provided at one end thereof and connected to the gas feeding ferrule 92A, which is a second ferrule of the endoscope 9, and a connector 32B provided at the other end thereof and connected to the gas feeding port 23 of the reprocessor 1. The gas feeding port 23 is in communication with the gas feeding ferrule 92A of the endoscope 9 via the second tube 32.

The reprocessor 1 includes the liquid pump 51, a gas pump 52 which is a compressor for feeding gas, a first solenoid valve 53, a second solenoid valve 54, and a controller 61.

The liquid pump 51 takes in liquid, such as cleaning solution in the processing tank 5, from the circulation port 16 having the mesh filter 15 into a conduit 55, pressurizes the taken-in liquid, and feeds the pressurized liquid to a branch conduit 59. Although not shown, a part of the liquid taken in from the circulation port 16 is fed to the processing tank 5 by another pump. The mesh filter 15 filters the contaminants P that flow down from the endoscope 9 and float in the liquid in the processing tank 5.

The liquid pump 51 is connected to the first solenoid valve 53 via the branch conduit 59. The first solenoid valve 53 is connected to the water supply port 22 via a conduit 57. A check valve 51A is disposed in an ejection conduit of the liquid pump 51.

The gas pump 52 takes in gas via a conduit 56, pressurizes the taken-in gas, and feeds the pressurized gas to the branch conduit 59. The gas is air, for example. The gas pump 52 is connected to the second solenoid valve 54 via the branch conduit 59. The second solenoid valve 54 is connected to the gas feeding port 23 via a conduit 58. A check valve 52A is provided in an ejection conduit of the gas pump 52. A pressure sensor 83 that detects a fluid pressure is disposed in the branch conduit 59.

The processing tank 5 is provided with a second sensor 82 that detects contaminants in the liquid stored in the tank. The second sensor 82 is a turbidity sensor or the like that measures a turbidity of the liquid. The turbidity sensor detects the contaminants P in the liquid by measuring light transmittance of the liquid. Since the stored liquid circulates through the conduit 90, when the contaminants P in the conduit 90 are removed, the contaminants P in the stored liquid are also reduced. In other words, the contaminants P detected by the second sensor 82 can be regarded as contaminants P in the conduit 90. The second sensor 82 may preferably be placed in the vicinity of the opening O93 of the endoscope 9 placed in the processing tank 5.

The controller 61 may be implemented by processing circuitry including one or more processors. For instance, the controller 61 includes processing circuitry including a CPU 62, which is a central processing unit of a computer, and a memory 63 including a ROM, a RAM, or the like. Functions of the controller 61 are realized by the CPU 62 reading and executing a program from the memory 63. A program for causing the computer to execute reprocessing, which is stored in the memory 63, is retained in a non-temporary computer-readable storage medium 8, and may be transferred to the memory 63. The memory 63 may store data corresponding to a model of the endoscope 9 obtained from an RFID tag provided on the endoscope 9, for example.

The controller 61 further includes connection interfaces 64. The controller 61 is electrically connected to the liquid pump 51, the gas pump 52, the first solenoid valve 53, the second solenoid valve 54, the first sensor 81, the second sensor 82, and the pressure sensor 83 via respective connection interfaces 64. Each connection interface 64 may include an electrical line and a connector through which control signals are transmitted between the controller 61 and a respective one of the connected elements such as the liquid pump 51, the gas pump 52, the first solenoid valve 53, the second solenoid valve 54, the first sensor 81, the second sensor 82, or the pressure sensor 83. The controller 61 may be implemented as a separate control apparatus connectable to the reprocessor 1 via the connection interfaces 64. In this case, the controller 61 may be operatively coupled to the reprocessor 1 in a wired or wireless manner, and the controller 61 and the reprocessor 1 may form an endoscope reprocessing system.

When supplying liquid, the controller 61 activates the liquid pump 51 and stops the gas pump 52. When supplying gas, the controller 61 stops the liquid pump 51 and activates the gas pump 52.

When the controller 61 activates the liquid pump 51 and then activates the gas pump 52, a gas-liquid mixture flow is supplied to the conduit 90. By repeatedly feeding air from the gas pump 52 after supplying liquid, the supplied fluid becomes the gas-liquid mixture flow in which liquid and gas are present in a mixed state. The gas-liquid mixture flow includes all of a state in which gas bubbles are present in liquid, a state in which liquid droplets are present in gas, and a state in which liquid slugs and gas pockets are present adjacent to one another.

The controller 61 controls an opening or closing state of the first solenoid valve 53 so that a fluid at a predetermined flow rate (predetermined pressure) is supplied to the first conduit 91 via the first tube 31. The controller 61 controls an opening or closing state of the second solenoid valve 54 so that a fluid at a predetermined flow rate is supplied to the second conduit 92 via the second tube 32.

The liquid pump 51, the gas pump 52, the first solenoid valve 53, and the second solenoid valve 54, the pressure sensor 83, and the like configure a fluid supply unit (hereinafter may be referred to as a fluid supply assembly) 10. The fluid supply unit 10 supplies fluid to each of the first tube 31 and the second tube 32. The controller 61 controls timing at which the fluid supply unit 10 supplies fluid to the conduit 90.

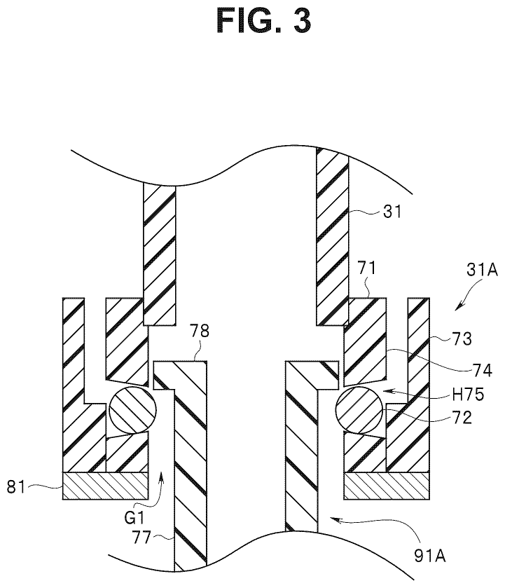

FIG. 3 is a cross-sectional view of a joint section between the first connector 31A of the first tube 31 and the forceps ferrule 91A of the endoscope 9. It should be noted that, description of a joint section between the second connector 32A of the second tube 32 and the gas feeding ferrule 92A of the endoscope 9 is omitted since this joint section has the same configuration as the joint section between the first connector 31A and the forceps ferrule 91A.

The first connector 31A includes a connector main body 71 provided at a distal end of the first tube 31, a plurality of spheres 72, and a connector cover 73 provided on an outer circumferential section of the connector main body 71.

The connector main body 71 is made of plastic or the like. The connector main body 71 is tubular, and includes a plurality of circular holes H75 in a circumferential side portion 74. For example, four holes H75 are provided in the circumferential side portion 74 of the connector main body 71 at equal intervals along a circumferential direction. Each of the holes H75 has a decreasing diameter from an outer surface toward an inner surface of the connector main body 71, and has a tapered cross section in a thickness direction of the circumferential side portion 74.

The plurality of spheres 72 are configured of metal or the like. The plurality of spheres 72 each has a diameter larger than that of each of the holes H75 in an inner circumferential surface of the circumferential side portion 74, and are placed with a part fitted in the holes H75, so that the spheres do not fall off from the inner circumferential surface of the circumferential side portion 74.

The connector cover 73 is configured of plastic or the like. The connector cover 73 is placed on an outside of the connector main body 71 where the plurality of spheres 72 are placed.

The forceps ferrule 91A is made of metal, resin, or the like. The forceps ferrule 91A has a body section 77 having a cylindrical shape, and includes an outwardly extending flange 78 at a distal end.

The connector 31A of the first tube 31 is detachably attached to the forceps ferrule 91A. In a state where the connector 31A is attached to the forceps ferrule 91A, the plurality (four, in this case) of spheres 72 of the connector 31A engage with the outwardly extending flange 78 of the forceps ferrule 91A to prevent detachment of the flange. A circumferential gap G1 is formed between the body section 77 of the forceps ferrule 91A and the connector main body 71.

The first sensor 81 is a contact sensor, an optical sensor, or the like that outputs a detection signal when the first connector 31A is connected to the forceps ferrule 91A.

The fluid fed from the first tube 31 is introduced to an inner side of the body section 77 of the forceps ferrule 91A, and flows out from the gap G1 via the space between the hole H75 and the sphere 72. The fluid flowing out from the gap G1 in a connection region between the connector 31A and the forceps ferrule 91A cleans an outer peripheral surface of the forceps ferrule 91A.

It should be noted that the shape of the connector 31A of the first tube 31 is not limited to that mentioned above, and a connector disclosed in International Publication No. WO 2015/001843 already described may also be employed.

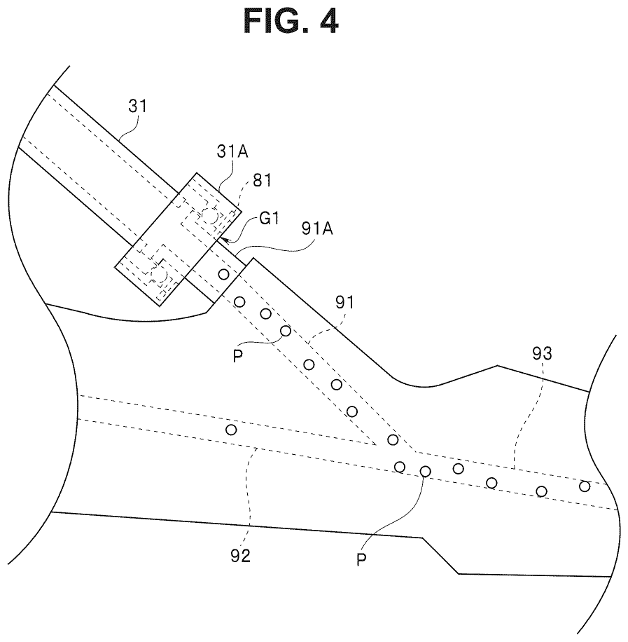

As shown in FIG. 4, the contaminants P may adhere to the conduit 90 of the endoscope 9 after use, particularly to the first conduit 91 and the third conduit 93, which are withdrawal paths for the treatment instrument inserted into the body.

As already described, there is the gap G1 between the first connector 31A of the first tube 31 connected to the forceps ferrule 91A and the forceps ferrule 91A. For this reason, in the reprocessing, for example, when the fluid supplied to the second conduit 92 flows back through the first conduit 91 via the merging section, the contaminants P flushed away by the fluid may be trapped in the gap G1.

As will be mentioned below, the controller 61 controls the timing at which the fluid supply unit 10 supplies fluid to the conduit 90. Specifically, the controller 61 controls the fluid supply unit 10 so that, when the first connector 31A is not connected to the forceps ferrule 91A, fluid is supplied to the first conduit 91 and the third conduit 93 via the second tube 32 and the second conduit 92 where the second connector 32A is connected to the gas feeding ferrule 92A.

The endoscope reprocessor includes a first tube including a first connector to be connected to a first ferrule of the endoscope, a second tube including a second connector to be connected to a second ferrule of the endoscope, a fluid supply unit that supplies fluid to a conduit of the endoscope via each of the first tube and the second tube, and a controller (processor) that controls the fluid supply unit.

The conduit includes a first conduit in communication with the first connector, a second conduit in communication with the second connector, and a third conduit where the first conduit and the second conduit merge.

The third conduit includes an opening at a distal end of an insertion portion of the endoscope. In the endoscope reprocessing, the controller controls the fluid supply unit so that, when the first connector is not connected to the first ferrule, the fluid is supplied to the first conduit and the third conduit via the second tube and the second conduit where the second connector is connected to the second ferrule.

By the above control of the controller 61, the contaminants P at the forceps ferrule 91A and the first conduit 91 are emitted together with the fluid from the forceps ferrule 91A. Since the endoscope reprocessor 1 does not require the user to brush clean the forceps ferrule 91A before reprocessing, efficient reprocessing can be performed.

### Method of Operating Endoscope Reprocessor

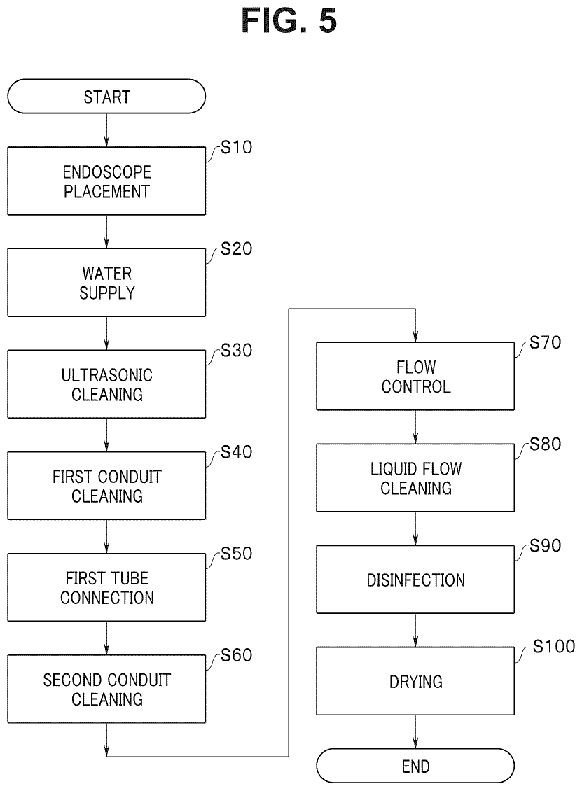

An example of a method of operating the reprocessor 1 will be described along the flowchart shown in FIG. 5.

### <Step S10> Endoscope Placement Step

The user opens the top cover 3 of the reprocessor 1 and sets the endoscope 9. Specifically, the user connects the second connector 32A of the second tube 32 to the gas feeding ferrule 92A of the endoscope 9, and connects the connector 32B to the gas feeding port 23.

The user also connects the connector 31B of the first tube 31 to the water supply port 22. However, the first connector 31A of the first tube 31 is not connected to the forceps ferrule 91A. It should be noted that the connection of the connector 31B of the first tube 31 to the water supply port 22 may be performed in a first tube connection step (S50) mentioned below.

After establishing connection between the reprocessor 1 and the endoscope 9, the user places the endoscope 9 in the endoscope placement section 11, and brings the top cover 3 into the closed state.

When the user gives a start instruction for predetermined reprocessing such as cleaning and disinfection from the operation panel 6, the CPU 62 reads a predetermined program from the memory 63 and starts a process of the program. To the memory 63, for example, a program for the endoscope reprocessor, which is stored in the non-temporary computer-readable storage medium 8, is transferred in advance.

### <Step S20> Water Supply Step

Based on a control signal from the CPU 62, water is supplied from the water supply nozzle 26 to the processing tank 5. When the water level in the processing tank 5 detected by the water level sensor 27 reaches a predetermined value, the water supply automatically stops.

### <Step S30> Ultrasonic Cleaning Step

When an unshown transducer disposed on a lower surface of the processing tank 5 is activated, ultrasonic waves are applied to the water stored in the processing tank 5. The ultrasonic cleaning cleans the contaminants P on an outer surface of the endoscope 9.

In step S10, the first connector 31A of the first tube 31 is not connected to the forceps ferrule 91A of the endoscope 9. When the controller 61 detects, based on a detection result of the first sensor 81, that the first connector 31A is not connected to the forceps ferrule 91A, the controller 61 proceeds to step S40.

It should be noted that the first sensor 81 is not an essential component. For example, the controller 61 may request the user to confirm that the first connector 31A is not connected to the forceps ferrule 91A, and after receiving a response from the user, proceed to step S40.

### <Step S40> First Conduit Cleaning Step

The controller 61 controls the fluid supply unit 10 so that, when the first tube 31 is in a state not connected to the forceps ferrule 91A, which is the first ferrule, water is supplied to the second conduit 92 via the second tube 32. The water supplied to the second conduit 92 flows not only to the third conduit 93, but also to the forceps ferrule 91A via the first conduit 91 from the merging section between the first conduit 91 and the second conduit 92.

Specifically, the controller 61 controls the fluid supply unit 10 to perform a first conduit cleaning step in which the second solenoid valve 54 is “opened”, and water is supplied to the second conduit 92 via the second tube 32.

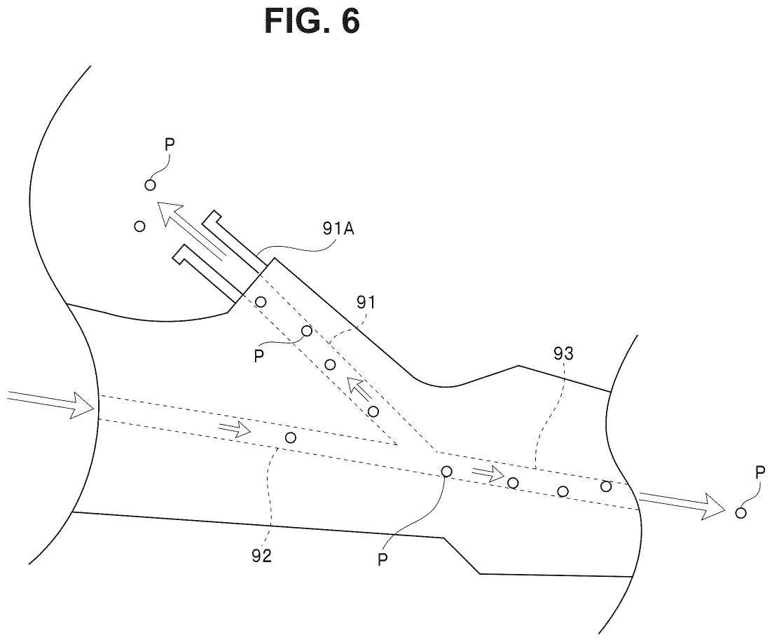

As shown in FIG. 6, in the first conduit cleaning step, when water flows into the third conduit 93, a part of the water flows back from the merging section to the first conduit 91. The contaminants P in the third conduit 93 are discharged together with the water from the opening O93 at the distal end. The contaminants P in the first conduit 91 and the forceps ferrule 91A are discharged together with the water from the forceps ferrule 91A.

When the turbidity of the water detected by the second sensor 82 becomes less than a predetermined value, the controller 61 determines that the contaminants P in the conduit 90 including the first conduit 91 have been substantially removed. Then, the controller 61 stops the first conduit cleaning step, and generates an instruction signal for notifying the user. The instruction signal is a signal to instruct that the first connector 31A of the first tube 31 be connected to the forceps ferrule 91A, which is the first ferrule. The instruction signal is, for example, at least one of a display on the operation panel 6 or a sound.

The second sensor 82 is an optical sensor that measures the turbidity of water passing through the conduit 90. The controller 61 may use a pressure sensor 83 instead of the second sensor 82 to determine a timing to end the first conduit cleaning step. In other words, when the contaminants P in the conduit 90 are removed, the pressure loss in the conduit 90 decreases, thereby lowering the water supply pressure detected by pressure sensor 83. The controller 61 may also determine the timing to end the first conduit cleaning step based on a water feeding rate detected by a flow rate sensor (not shown). Contaminants in the water may be detected by a conductivity measurement sensor. The controller 61 may determine the timing to end the first conduit cleaning step by comprehensively using output results of a plurality of different kinds of sensors.

The controller 61 may automatically stop the first conduit cleaning step after elapse of a predetermined time after starting the first conduit cleaning step, and generate the instruction signal. In other words, the timing to end the first conduit cleaning step is determined by the controller 61 based on a detection result of at least one of the sensors or based on time.

### <Step S50> First Tube Connection

Upon confirming the notification based on the instruction signal, the user opens the top cover 3 of the reprocessor 1, and connects the first connector 31A of the first tube 31 to the forceps ferrule 91A of the endoscope 9. Then, the user brings the top cover 3 into the closed state.

The connection of the first tube (connection of the first connector 31A to the forceps ferrule 91A) may be performed automatically. For example, a robot arm disposed on the reprocessor 1 or a robot placed in the vicinity of the reprocessor 1 may connect the first tube 31 to the forceps ferrule 91A.

At the time of ending the first conduit cleaning step, the controller 61 may decrease the rate of water fed to the second conduit 92 rather than stopping the supply of water to the second conduit 92. In other words, the first connector 31A may be connected to the forceps ferrule 91A in a state where a small amount of water is flowing back from the first conduit 91 to the forceps ferrule 91A.

When the controller 61 detects, based on a detection result of the first sensor 81, that the first connector 31A is connected to the forceps ferrule 91A, the controller 61 proceeds to step S60. The controller 61 may request the user to confirm that the first connector 31A is connected to the forceps ferrule 91A, and after receiving a response from the user, proceeds to step S60.

### <Step S60> Second Conduit Cleaning Step

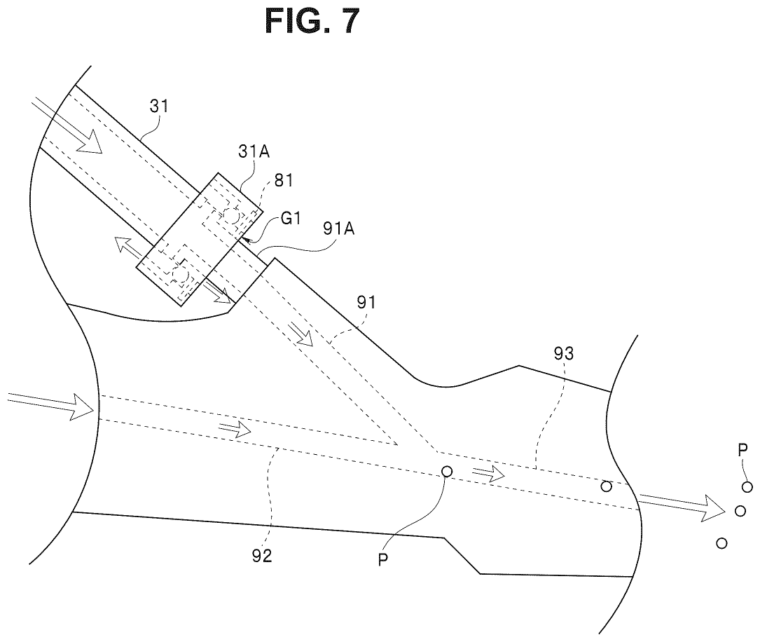

The controller 61 performs a second conduit cleaning step in which the first solenoid valve 53 is also “opened”, and water is supplied to the conduit 90 via the first tube 31 and the second tube 32. As shown in FIG. 7, in the second conduit cleaning step, water is supplied to the first conduit 91 and the second conduit 92, and the supplied water is emitted from the opening O93 via the third conduit 93.

The water supplied from the first tube 31 flows out from the gap G1 in the connection region between the connector 31A and the forceps ferrule 91A, to clean the outer peripheral surface of the forceps ferrule 91A.

The controller 61 ends the second conduit cleaning step based on an output result of the second sensor 82, elapse of time, or the like.

It should be noted that, in the second conduit cleaning step, there may be a period of time in which the second solenoid valve 54 is “closed” and the feeding of liquid to the second tube 32 is stopped. There may be a period of time in which the liquid pump 51 and the gas pump 52 are activated, and a gas-liquid mixture flow is supplied to the conduit 90. There may be a period of time in which the second connector 32A is detached from the gas feeding ferrule 92A.

After step S60 (second conduit cleaning step), the first connector 31A may be detached from the forceps ferrule 91A, and steps from step S40 (first conduit cleaning step) may be repeated again.

### <Step S70> Flow Control Step

A flow control step is a step in which a flow rate of liquid is measured and compared with a normal flow rate, to thereby confirm that the tubes are correctly connected.

### <Step S80> Liquid Flow Cleaning Step

The cleaning solution in a cleaning solution tank is poured from the cleaning solution nozzle 24 into the processing tank 5 in which water is stored. The cleaning solution diluted with the water is discharged from the circulation port 16 by the liquid pump 51, and thereby supplied again to the processing tank 5 and the conduit 90.

### <Step S90> Disinfection Step

After the diluted cleaning solution is discharged from the processing tank 5, a disinfectant solution in a disinfectant solution tank is poured from the disinfectant solution nozzle 25 into the processing tank 5. The disinfectant solution is sucked in from the circulation port 16 by the liquid pump 51, and thereby supplied again to the processing tank 5 and the conduit 90.

<Step S100> Drying Step

After the disinfectant solution is discharged from the processing tank 5, air is fed into the conduit 90 by the gas pump 52 to perform a drying process to remove water from the conduit. A drying liquid such as unshown alcohol may be fed to the conduit 90.

The above completes the reprocessing of the endoscope 9 placed in the processing tank 5. It should be noted that the reprocessing is not limited to the processing mentioned above. For example, a rinsing step using water and a drying step may be performed between each step. The flow control step (step S70) may be omitted.

As described above, in the method of operating the endoscope reprocessor, the second connector is connected to the second ferrule, the fluid is supplied to the first conduit and the third conduit where the first connector is not connected to the first ferrule, the first connector is connected to the first ferrule, and the fluid is supplied to the third conduit via the first conduit and the second conduit.

According to the method of operating the endoscope reprocessor as mentioned above, the contaminants P can be prevented from being trapped in the space between the ferrule of the endoscope 9 and the connector of the tube, without the need to perform preliminary brush cleaning, thereby causing the endoscope reprocessing to be efficiently performed.

A program for operating the endoscope reprocessor causes a computer to execute control so that, when the first connector 31A is not connected to the forceps ferrule 91A (first ferrule), the fluid is supplied to the first conduit 91 and the third conduit 93 via the second tube 32 and the second conduit 92 where the second connector 32A is connected to the gas feeding ferrule 92A (second ferrule).

The endoscope 9 according to the embodiment is a flexible endoscope for medical use, but the endoscope of the present disclosure may be a rigid endoscope, and the application thereof may be industrial. A monitor (not shown) may be directly connected to the operation portion 9B of the endoscope 9.

The present disclosure is not limited to the above-mentioned embodiment, etc., and various changes, modifications, etc. can be made without departing from the gist of the present disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope reprocessor configured to reprocess an endoscope,
the endoscope comprising:
   a conduit including a first conduit, a second conduit, and a third conduit, the first conduit, the second conduit, and the third conduit each opening at one end side and merging at a merging point on another end side, and
the endoscope reprocessor comprising:
   a first tube including a first connector configured to be connected to a first ferrule, the first ferrule being in communication with the first conduit of the endoscope;
   a second tube including a second connector configured to be connected to a second ferrule, the second ferrule being in communication with the second conduit of the endoscope;
   a fluid supply unit configured to supply fluid to the conduit of the endoscope via each of the first tube and the second tube;
   a sensor configured to measure the fluid; and
   a processor configured to control the fluid supply unit, wherein
the processor is configured to:
   carry out a first cleaning step of, when the first connector is not connected to the first ferrule, causing the fluid supply unit to supply the fluid to the first conduit and the third conduit via the second tube and the second conduit where the second connector is connected to the second ferrule;
   determine a timing to end the first cleaning step based on a detection signal of the sensor; and
   carry out a second cleaning step of causing the fluid supply unit to supply the fluid to the first tube and the second tube.

Clause 2: The endoscope reprocessor according to clause 1, further comprising:
a first sensor configured to detect connection of the first connector to the first ferrule, wherein
the processor is configured to control the fluid supply unit based on a detection result of the first sensor.

Clause 3: The endoscope reprocessor according to clause 2, wherein the first sensor is disposed at the first connector.

Clause 4: The endoscope reprocessor according to clause 1, wherein
the sensor is a second sensor configured to detect contaminants in the conduit, and
the processor is configured to generate, when the contaminants detected by the second sensor are less than a predetermined value, an instruction signal for connecting the first connector to the first ferrule.

Clause 5: The endoscope reprocessor according to clause 4, wherein the second sensor is an optical sensor configured to measure a turbidity of the fluid passing through the conduit.

Clause 6: The endoscope reprocessor according to clause 4, wherein the instruction signal is a display or a sound for notifying a user.

Clause 7: The endoscope reprocessor according to clause 1, wherein the first connector includes a hole through which the fluid leaks out from a connection region between the first connector and the first ferrule to an outer peripheral surface of the first ferrule.

Clause 8: The endoscope reprocessor according to clause 6, wherein the first ferrule is a forceps ferrule, and the second ferrule is a gas feeding ferrule.

Clause 9: A method of operating an endoscope reprocessor for an endoscope, the endoscope comprising:
a first conduit in communication with a first ferrule;
a second conduit in communication with a second ferrule; and
a third conduit where the first conduit and the second conduit merge, the third conduit including an opening at a distal end of an insertion portion of the endoscope, and
the method comprising:
   carrying out a first cleaning step of, when a first connector of a first tube is not connected to the first ferrule, supplying fluid to the first conduit and the third conduit via the second tube and the second conduit where a second connector of a second tube is connected to the second ferrule;
   determining a timing to end the first cleaning step based on a detection signal of a sensor configured to measure the fluid; and
   carrying out a second cleaning step of supplying the fluid to the first tube and the second tube.

Clause 10: The method of operating the endoscope reprocessor according to clause 9, the method further comprising:
controlling a fluid supply unit based on a detection result of a first sensor, the fluid supply unit being configured to supply fluid to the conduit of the endoscope, and the first sensor being configured to detect connection of the first connector to the first ferrule.

Clause 11: The method of operating the endoscope reprocessor according to clause 10, wherein the first sensor is disposed at the first connector.

Clause 12: The method of operating the endoscope reprocessor according to clause 9, wherein
the sensor is a second sensor configured to detect contaminants in the conduit, and
the method further comprises generating, when the contaminants detected by the second sensor are less than a predetermined value, an instruction signal for connecting the first connector to the first ferrule.

Clause 13: The method of operating the endoscope reprocessor according to clause 12, wherein the second sensor is an optical sensor configured to measure a turbidity of the fluid passing through the conduit.

Clause 14: The method of operating the endoscope reprocessor according to clause 12, wherein the instruction signal is a display or a sound for notifying a user.

Clause 15: The method of operating the endoscope reprocessor according to clause 9, wherein the first connector includes a hole through which the fluid leaks out from a connection region between the first connector and the first ferrule to an outer peripheral surface of the first ferrule.

Clause 16: The method of operating the endoscope reprocessor according to clause 14, wherein the first ferrule is a forceps ferrule, and the second ferrule is a gas feeding ferrule.

Clause 17: A control apparatus for controlling an endoscope reprocessor for an endoscope,
the control apparatus comprising a processor,
the endoscope comprising:
   a first conduit in communication with a first ferrule;
   a second conduit in communication with a second ferrule; and
   a third conduit where the first conduit and the second conduit merge, the third conduit including an opening at a distal end of an insertion portion of the endoscope, and
the processor being configured to:
   carry out a first cleaning step of, when a first connector of a first tube is not connected to the first ferrule, causing the endoscope reprocessor to supply fluid to the first conduit and the third conduit via the second tube and the second conduit where a second connector of a second tube is connected to the second ferrule;
   determine a timing to end the first cleaning step based on a detection signal of a sensor configured to measure the fluid; and
   carry out a second cleaning step of causing the endoscope reprocessor to supply the fluid to the first tube and the second tube.

Clause 18: The control apparatus according to clause 17, wherein the processor is configured to control a fluid supply unit based on a detection result of a first sensor, the fluid supply unit being configured to supply fluid to the conduit of the endoscope, and the first sensor being configured to detect connection of the first connector to the first ferrule.

Clause 19: The control apparatus according to clause 18, wherein the first sensor is disposed at the first connector.

Clause 20: The control apparatus according to clause 17, wherein
the sensor is a second sensor configured to detect contaminants in the conduit, and
the processor is configured to generate, when the contaminants detected by the second sensor are less than a predetermined value, an instruction signal for connecting the first connector to the first ferrule.

What is claimed is:

1. An endoscope reprocessor comprising:
a first tube connectable to a first ferrule of an endoscope;
a second tube connectable to a second ferrule of the endoscope;
a fluid supply assembly configured to supply a fluid to an internal conduit of the endoscope via the first and second tubes, the internal conduit including a first conduit communicating with the first ferrule, a second conduit communicating with the second ferrule, and a third conduit, each of the first to third conduits being open at one end and merging with the other two conduits at a merging point at another end;
a sensor configured to detect a property of the fluid; and
a processor configured to:
perform a first cleaning process to cause the fluid supply assembly to supply, when the first tube is not connected to the first ferrule, the fluid to the first conduit and the third conduit via the second conduit and the second tube connected to the second ferrule;
determine an end timing of the first cleaning process based on a detection signal from the sensor; and
perform a second cleaning process to cause the fluid supply assembly to supply the fluid to the first and second tubes.

2. The endoscope reprocessor according to claim 1, further comprising a connection sensor configured to detect a connection of a first connector of the first tube to the first ferrule, wherein
the processor is further configured to control the fluid supply assembly based on a detection result of the connection sensor.

3. The endoscope reprocessor according to claim 2, wherein the connection sensor is disposed at the first connector of the first tube.

4. The endoscope reprocessor according to claim 1, wherein
the sensor is further configured to detect a contamination level in the conduit, and
the processor is further configured to provide an instruction signal for connecting a first connector of the first tube to the first ferrule when the contamination level detected by the sensor is less than a predetermined value.

5. The endoscope reprocessor according to claim 4, wherein the sensor includes an optical sensor configured to measure a turbidity of the fluid having passed through the internal conduit.

6. The endoscope reprocessor according to claim 4, wherein the instruction signal is provided as at least one of a display or a sound for user notification.

7. The endoscope reprocessor according to claim 1, wherein
the first tube includes a first connector connectable to the first ferrule, and
the first connector includes a hole configured to allow the fluid to leak out from a connection region between the first connector and the first ferrule to an outer peripheral surface of the first ferrule.

8. The endoscope reprocessor according to claim 1, wherein the first ferrule is a forceps ferrule, and the second ferrule is a gas supply ferrule.

9. A control method implementable by an endoscope reprocessor comprising a first tube connectable to a first ferrule of an endoscope, and a second tube connectable to a second ferrule of the endoscope, the control method comprising:
performing a first cleaning process to supply, when the first tube is not connected to the first ferrule, a fluid to a first conduit and a third conduit via a second conduit and the second tube connected to the second ferrule, wherein the endoscope comprises an internal conduit including the first conduit communicating with the first ferrule, and the second conduit communicating with the second ferrule, the first and second conduits converging into the third conduit having an opening at a distal end of an insertion portion of the endoscope;
determining an end timing of the first cleaning process based on a detection signal from a sensor configured to detect a property of the fluid; and
performing a second cleaning process to supply the fluid to the first and second tubes.

10. The control method according to claim 9, further comprising:
controlling a fluid supply assembly configured to supply the fluid to the internal conduit of the endoscope, based on a detection result of a connection sensor configured to detect a connection of a first connector of the first tube to the first ferrule.

11. The control method according to claim 10, wherein the connection sensor is disposed at the first connector of the first tube.

12. The control method according to claim 9, wherein
the sensor is further configured to detect a contamination level in the conduit, and
the control method further comprises providing an instruction signal for connecting a first connector of the first tube to the first ferrule when the contamination level detected by the sensor is less than a predetermined value.

13. The control method according to claim 12, wherein the sensor includes an optical sensor configured to measure a turbidity of the fluid having passed through the internal conduit.

14. The control method according to claim 12, wherein the instruction signal is provided as at least one of a display or a sound for user notification.

15. The control method according to claim 9, wherein
the first tube includes a first connector connectable to the first ferrule, and
the first connector includes a hole configured to allow the fluid to leak out from a connection region between the first connector and the first ferrule to an outer peripheral surface of the first ferrule.

16. The control method according to claim 9, wherein the first ferrule is a forceps ferrule, and the second ferrule is a gas supply ferrule.

17. A control apparatus comprising:
a connection interface connectable to a fluid supply assembly configured to supply a fluid to an internal conduit of an endoscope via a first tube and a second tube, wherein the endoscope comprises a first ferrule connectable to the first tube, a second ferrule connectable to the second tube, and the internal conduit including a first conduit communicating with the first ferrule, and a second conduit communicating with the second ferrule, the first and second conduits converging into a third conduit having an opening at a distal end of an insertion portion of the endoscope; and
a processor configured to:
perform a first cleaning process to cause the fluid supply assembly to supply, when the first tube is not connected to the first ferrule, a fluid to the first conduit and the third conduit via the second conduit and the second tube connected to the second ferrule;
determine an end timing of the first cleaning process based on a detection signal from a sensor configured to detect a property of the fluid; and
perform a second cleaning process to cause the fluid supply assembly to supply the fluid to the first and second tubes.

18. The control apparatus according to claim 17, wherein
the first tube includes a first connector connectable to the first ferrule, and
the processor is further configured to control the fluid supply assembly based on a detection result of a connection sensor configured to detect a connection of the first connector of the first tube to the first ferrule.

19. The control apparatus according to claim 18, wherein the connection sensor is disposed at the first connector of the first tube.

20. The control apparatus according to claim 17, wherein
the sensor is further configured to detect a contamination level in the conduit, and
the processor is further configured to provide an instruction signal for connecting a first connector of the first tube to the first ferrule when the contamination level detected by the sensor is less than a predetermined value.
